(19) United States
(12) Patent Application Publication
(10) Pub. No.: US 2018/0056048 A1
(43) Pub. Date: Mar. 1, 2018

(54) DRAINAGE DEVICE

(71) Applicant: Ramiro Manuel Mineiro Guedes de Campos, Rio de Janeiro - RJ (BR)

(72) Inventor: Ramiro Manuel Mineiro Guedes de Campos, Rio de Janeiro - RJ (BR)

(21) Appl. No.: 15/690,549

(22) Filed: Aug. 30, 2017

(30) Foreign Application Priority Data
Aug. 30, 2016 (BR) BR202016020046-1

(51) Int. Cl. A61M 27/00; A61M 1/00; A61M 25/00

(52) U.S. Cl. CPC A61M 27/00; A61M 2025/0004; A61M 25/0028; A61M 1/0086

(57) ABSTRACT

The present invention refers to a drainage device comprising an outer tube; an inner tube, concentrically inserted into the outer tube; wherein the front portions of the outer and inner tubes are inserted into the patient's body, and wherein the rear portions of the outer and inner tubes are external to the patient's body; wherein the outer tube comprises a plurality of holes in its front portion; wherein an aperture in the rear portion of the inner tube is connected to a suction device. The drainage device consists of a single injectable piece, wherein the inner tube and the outer tube are connected by at least two side guides located in the front portion of the inner tube; wherein the front portion of the outer tube has an aperture in direct contact with the site to be drained; the rear portion of the outer tube has an aperture in direct contact with the environment.

### CROSS-REFERENCE TO RELATED APPLICATIONS

This application claims the benefit of Brazil Patent Application No. BR202016020046-1 filed Aug. 30, 2016, the entire disclosure of which is incorporated herein by reference.

The present invention refers to a drainage device, designed to be used in post-surgical procedures for the efficient removal of fluids, without risk, giving greater comfort and mobility to the patient.

### DESCRIPTION OF THE PRIOR ART

The use of drainage devices to assist in scarring in surgical procedures is already widely known in the state of the art. The use of these devices allows the accumulated fluid/gas or air to flow out of the area where the surgical procedure was performed, preventing the excessive accumulation of fluid close to the surgical scar, reducing the risks of infections in the affected area, increasing the chances of a better scarring, and avoiding additional surgical measures.

There are two types of common drainage devices in the prior art: the laminar and the tubular ones. The laminar drainage devices are usually made of silicone, i.e., malleable and soft material and are commonly called Penrose drains.

The main advantage of this type of drain is that since it is malleable, it accommodates itself to the interior of the body without causing damage and, given its almost inert material, causes few inflammatory reactions, being able to remain in the body for a longer period of time. However, since it is made of a very malleable material, when in use, this drain tends to bind together, i.e., its tubular structure is compressed, due to the low pressure created inside the drain, forming a blade, causing it to stop working.

Furthermore, since this drain is used by a suction device, structures and tissues adjacent the region where the drainage device is located also tend to be sucked in such a way that they close the drain openings, clogging it.

The tubular drainage devices are made of a less flexible material when compared to the laminar drainage devices, and this greater rigidity prevent their tubular structure to bind together. This type of drainage device can be made of a single tube, double or triple, and must have a plurality of side holes in its front portion.

However, the materials used for manufacturing this type of more rigid drain such as, for example, latex, have a greater tendency to trigger inflammatory reactions because they are not an inert material and therefore cannot remain in the body for a long periods of time. Moreover, since they are less flexible, they can cause discomfort to the patient and damage the tissues and vessels around the drainage device.

Furthermore, the U.S. Pat. No. 3,528,427 discloses a surgical drain catheter with a tubular cannula arranged in the inside and a casing, which is also tubular, thus, forming a drainage device. It is also described in this document that a first set of holes is provided near the front portion of the cannula, which has an aperture. The front portion of the casing also includes a plurality of holes that are usually smaller than the holes of the cannula.

However, this drainage device is made of a rigid material making it impossible for the patient to move and, therefore, it is recommended only for controlled environments because, as indicated above, the most rigid tube may cause tissue damage around the device, hindering scarring of the surgery area. Furthermore, the tip of the front portion of this tube is opened, facilitating drain clogging and reducing drainage efficiency.

Moreover, the cannula and the casing are separate tubes, and are only engaged by their front ends, so that there is a great risk of both separating when the device is in use.

The U.S. Pat. No. 6,299,593 discloses a drainage device consisting of an inner tube and an outer tube, both containing a plurality of holes in its front ends. The outer and inner tubes are separated by septa to prevent collapse of the tubes.

However, the outer tube of this device has a square-shaped cross section, forming edges, so that, when replaced/removed from the surgical area there is a risk that these edges will cut or injure the tissues adjacent to the area, which may compromise the scarring of the area.

Moreover, the device of U.S. Pat. No. 6,299,593 has non-fixed parts, such as a solid plastic material, used to fill the space between the rear ends of the inner and outer tubes. These non-fixed parts can come loose from the drainage device, creating a risk to the patient.

### OBJECTIVES OF THE INVENTION

The objective of the present invention is, inter alia, to solve the above-mentioned technical issues, by providing a drainage device designed so that there is no risk of detachment of parts or any of the constituent parts, with greater resistance to binding together, and allowing greater comfort and mobility to the patient.

### BRIEF DESCRIPTION OF THE INVENTION

The objectives of the invention are achieved by means of a drainage device comprising: an outer tube, an inner tube concentrically inserted into the outer tube, the front portions thereof being inserted in the patient's body, and the posterior portions thereof being external to the patient's body.

Furthermore, the outer tube comprises, in its front portion, a plurality of holes, and the inner tube comprises an aperture in its posterior portion, which is connected to a suction device.

Also, the drainage device is formed by a single injectable piece, wherein the inner tube and the outer tube are connected by at least two side guides located in the front portion of the inner tube.

Furthermore, the front portion of the outer tube has an aperture in direct contact with the site to be drained and the posterior portion has an aperture in direct contact with the environment.

### BRIEF DESCRIPTION OF THE DRAWINGS

The present invention will now be described in greater detail with reference to an example of embodiment represented in the drawings. The figures show:

FIG. 1—sectional side view of the drainage device of the present invention;

FIG. 2—enlarged side view of the front portion of the drainage device of the present invention;

FIG. 3—front perspective view of the drainage device of the present invention; and

FIG. 4—rear perspective view of the drainage device of the present invention.

### DETAILED DESCRIPTION OF THE FIGURES

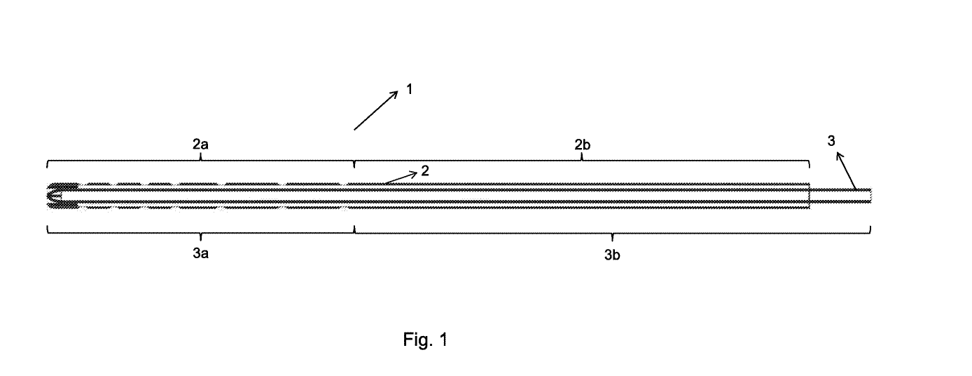

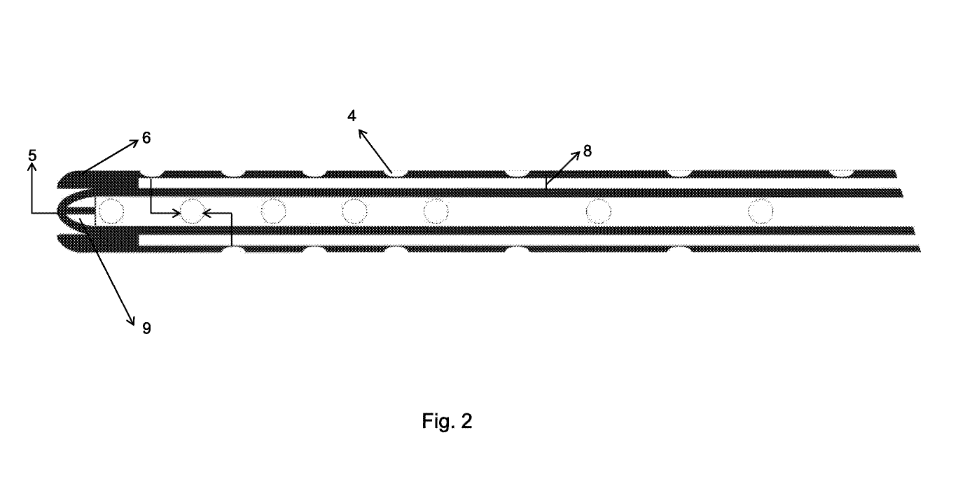

FIGS. 1 and 2 show a preferred embodiment of the present invention, showing a drainage device 1 consisting of an outer tube 2 and an inner tube 3, wherein the inner tube 3 is concentrically inserted into the outer tube 2.

The outer tube 2 comprises, in its front portion 2a, a plurality of holes 4 along ⅓ of its total length, ending before the end of its front portion. As shown in FIG. 2, the plurality of holes 4 is arranged in an alternated manner, i.e., two vertically opposed holes formed a 90 degree angle with the next two holes, and are at a distance of approximately 10 mm, so there is no weakening of the outer tube structure 2b in this area.

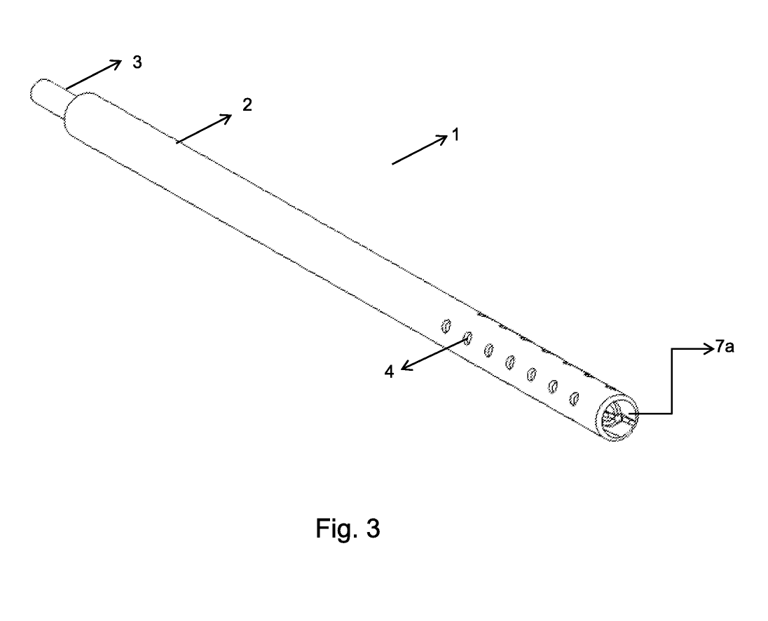

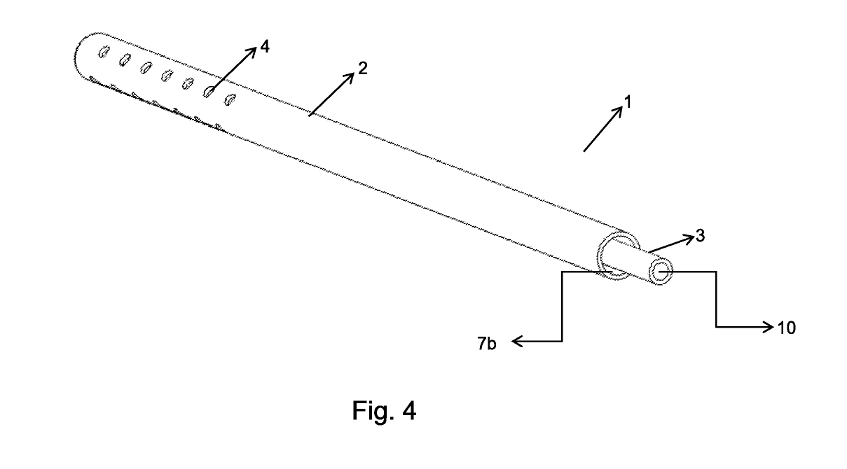

Moreover, as shown in FIGS. 3 and 4, the outer tube 2 has an opened front end 7a and rear end 7b. Similarly, the front portion 3a of the inner tube 3 has an aperture 9; however, the aperture 9 is covered by at least two arcuate fins, which form a curved cover 5 (FIG. 2), in a dome shape.

This curved cover 5 functions as a filter, so that when the device is in use, the cover 5 prevents elements from the aspirated fluid from entering the tube, thus preventing any clogging.

Furthermore, the inner tube 3 has, projected towards its front portion 3a, at least two side guides 6. These guides 6 fasten the inner tube 3 to the outer tube 2, in addition to maintain the inner tube 3 central to the outer tube.

It is noteworthy that, although the outer and inner tubes 2, 3 of the drainage device 1 are held together by the guides 6, the device 1 is formed of a single injected piece, without gluing or back welds, so that the detachment of any of the parts of the drainage device into the cavity to be drained is avoided, bringing greater safety to the patient.

The rear portion 3b of the inner tube 3, further, has an aperture 10 which is connected to a suction device, such as a vacuum pump or a syringe (not shown).

Thus, prior to actuation of the suction device, the drainage device 1 is inserted into the patient's body through a small incision as close and rectilinear as possible to the location where drainage is required, so that the front portion 2a of the outer tube 2 of the drainage device 1 is inserted into the patient's body, enabling the opening 7a to be in direct contact with the site to be drained.

Upon actuation of the suction device, connected to the rear portion 3b of the inner tube 3, a region of low pressure (pressure below atmospheric pressure) is formed inside the inner tube 3, so that the fluid suction process existing in the cavity to be drained is started, where the fluid to be drained will enter the inner tube 3 through the aperture 9 covered by the cover 5, which will function as a filter.

Simultaneously, the fluid will also enter the outer tube 2, through the aperture 7a and the plurality of holes 4. Since the rear portion of the outer tube 2 has an aperture 7b which will assist in balancing the atmospheric pressure within the patient's body, once the posterior portion 2b is external to the body and is in contact with the environment, allowing air flow to be created in a space 8 formed between the outer tube 2 and the inner tube 3.

Hence, the air flow will push the fluid aspirated by the plurality of holes 4 and aperture 7a and that is accumulated in space 8, for the inside of the inner tube 3 through the plurality of holes 4b, unclogging the space 8 and, consequently, the plurality of holes 4. Hence, this configuration allows a greater flow to pass through the drainage device 1, performing a faster and more efficient drainage.

Furthermore, since aperture 7b maintain the atmospheric pressure in the site to be drained, this prevents binding together of the drainage device 1 by maintaining a constant equilibrium of the atmospheric pressure within the patient's body.

Moreover, by maintaining atmospheric pressure at the drainage site, the aperture 7b, also prevents structures and tissues adjacent to the region to be drained from being sucked by the drainage device 1, closing its opening and interrupting the drainage of the region.

Lastly, this configuration, which is more resistant to binding together and clogging, makes possible to manufacture the drainage device in a more flexible material, such as flexible silicone. The drainage device 1 made of this material enables the patient to move, without moving the device 1 away from the drainage region, providing more comfort and mobility to the patient.

Although only one preferred embodiment has been described, it must be understood that the scope of the present invention covers other possible alternatives and that it is limited only by the content of the attached claim, the possible equivalents being included therein.

1. A drainage device comprising:
an outer tube;
an inner tube, concentrically inserted into the outer tube;
wherein the front portions of the outer and inner tubes are inserted into the patient's body, and
wherein the rear portions of the outer and inner tubes are external to the patient's body;
wherein the outer tube comprises a plurality of holes in its front portion;
wherein an aperture in the rear portion of the inner tube is connected to a suction device;
the drainage device consists of a single injectable piece, wherein the inner tube and the outer tube are connected by at least two side guides located in the front portion of the inner tube; wherein
the front portion of the outer tube has an aperture in direct contact with the site to be drained; and
the rear portion of the outer tube has an aperture in direct contact with the environment.
